(19) United States
(12) Patent Application Publication
(10) Pub. No.: US 2026/0294526 A1
(43) Pub. Date: Oct. 1, 2026

(54) DEVICE AND SYSTEMS FOR CREATING A VARIABLE FLOW INTERATRIAL SHUNT

(71) Applicant: Medtronic, Inc., Minneapolis, MN (US)

(72) Inventors: John B. Horrigan, Beverly, MA (US); Brian D. Pederson, East Bethel, MN (US); Kevin L. Sack, New Brighton, MN (US)

(21) Appl. No.: 19/474,836

(22) PCT Filed: Apr. 16, 2024
(86) PCT No.: PCT/US2024/024792

§ 371 Date: Oct. 13, 2025

Related U.S. Application Data
(60) Provisional application No. 63/497,161, filed on Apr. 19, 2023.

(51) Int. Cl. A61B 18/14; A61B 17/00; A61B 18/00; A61B 18/18; A61B 90/00

(52) U.S. Cl. CPC A61B 18/1492; A61B 18/1815; A61B 90/39; A61B 2017/00867; A61B 2018/00083; A61B 2018/00184; A61B 2018/00351; A61B 2018/00577; A61B 2018/00601; A61B 2018/0072; A61B 2018/144; A61B 2018/1861; A61B 2090/3966

(57) ABSTRACT

A medical device and method are described for creating a biostable shunt in a septal wall between a right atrium and a. left atrium of a heart. The medical device, such as an ablation device that includes an active portion, is advanced toward a septal wall of the heart such that the active portion is at least partially deflected by the septal wall. An electrical current is delivered to the active portion to provide an ablation of the septal wall from a cut, so as to form a single flap comprising the biostable shunt. The biostable shunt may provide variable flow based on the pressure differential between the right and left atrium.

This application is an international application with provisional priority of U.S. Provisional Patent Application No. 63/497,161, filed 19 Apr. 2023, the entire contents of which is incorporated herein by reference.

### FIELD

This disclosure generally relates to medical devices and, more particularly, to medical devices and associated techniques for forming shunts.

### BACKGROUND

Hypertension, which may lead to heart failure, may be the result of a higher-than-normal blood pressure. Interatrial shunting is currently being studied to treat heart failure and left atrial hypertension. A heart of a patient with heart failure may not efficiently pump blood, which may cause a pressure build-up in either the left atrium or right atrium, and may also cause fluid to be pushed into the lungs, resulting in pulmonary edema. Patients experiencing heart failure currently have limited treatment options.

Interatrial shunting is a technique for decompressing the left or right atria in patients suffering from heart failure. During this procedure, a blood flow pathway is created between the right atrium and the left atrium such that blood flows between the two atria.

There are two types of interatrial shunts. The first type is an implant-based shunt, which leaves a durable implant in the septal wall to maintain patency. In a typical procedure, the septal wall separating the atria is cut with a puncturing device. A mechanical device, such as a stent, is left in place to prevent tissue overgrowth and to maintain the shunt. The second type of interatrial shunt, a non-implant shunt, relies on cutting or ablating the septal wall tissue. Conventional shunting procedures may result in tissue overgrowth, thereby reducing the effectiveness of the shunt.

### SUMMARY

The present disclosure describes systems, devices, and techniques for creating a fluid pathway, or shunt, between a left atrium and a right atrium of a mammalian heart. The shunt can be maintained without the use of an implant, e.g., such as a stent. The shunt can be used, for example, to treat patients having heart failure and/or pulmonary edema.

Patients may experience different left atrial to right atrial pressure differentials depending on their activity levels (sleep, rest, walking, or exercising). A fixed-size interatrial shunt may be unable to vary interatrial flow in such conditions. A variable flow shunt formed according to the techniques of this disclosure, however, may advantageously enable interatrial flow to vary, which may provide a more physiological mechanism to relieve atrial hypertension.

In some examples described herein, a medical device includes a catheter defining a lumen; and an electrosurgical device movable within the lumen and configured to extend distally outward from a distal end of the catheter, the electrosurgical device comprising at least one electrically conductive wire having an active portion configured to cut septal wall tissue when an electrical current is applied to the wire; wherein the active portion is configured to provide a cut so as to form a single flap comprising a biostable valvular shunt in the septal wall tissue. In some examples, the valvular shunt is rendered biostable by inhibiting overgrowth, scarring, and/or reattachment of a removed portion of the ablated septal wall tissue to reduce and/or prevent the valvular shunt from closing. In some examples, the active portion is configured to form a U shape, a V shape, a C shape, a horseshoe shape, a parabolic shape, a square shape, and/or an arc shape.

In some examples, this disclosure describes a method of creating a biostable shunt in a septal wall between a left atrium and a right atrium of a heart. The method comprises advancing an ablation device towards the septal wall, the ablation device including an active portion, wherein the active portion is at least partially deflected by the septal wall; and delivering an electrical current to the active portion to provide an ablation of the septal wall from a cut, so as to form a single flap comprising the biostable shunt. In some examples, the active portion is configured to form a U shape, a V shape, a C shape, a horseshoe shape, a parabolic shape, a square shape, and/or an arc shape.

In other examples, the systems, devices, and techniques described herein can be used to create a shunt between two other hollow anatomical structures of a patient and to treat other patient conditions. Thus, while a shunt between a left atrium of a heart of a patient and a right atrium of the patient is primarily referred to herein, the systems, devices, and techniques can be used to form shunts in other locations of the heart, other locations of the body of a patient, or for other medical procedures in other examples.

The details of one or more examples of the techniques of this disclosure are set forth in the accompanying drawings and the description below. Other features, objects, and advantages of the techniques will be apparent from the description and drawings, and from the claims.

### BRIEF DESCRIPTION OF DRAWINGS

FIG. 1 is a perspective view depicting an example medical device configured to form a shunt at a target treatment site of a mammalian heart.

FIGS. 2A-2F illustrate an example technique for forming a biostable valvular shunt between a right atrium and a left atrium of a mammalian heart using the medical device of FIG. 1.

FIGS. 3A-3D comprise a set of graphs showing illustrative relationships between open cross-sectional area versus pressure gradient and material thickness for each of a plurality of biostable valvular shunts according to example embodiments.

FIG. 4A-4D comprise a set of flow curves showing projected flow profiles for the biostable valvular shunts shown in FIGS. 3A-3D, respectively, as compared against fixed circular holes with diameters of 5 mm and 8 mm.

FIG. 5 is a flowchart illustrating an example technique for forming a biostable valvular shunt between a right atrium and a left atrium of a mammalian heart using the medical device of FIG. 1.

### DETAILED DESCRIPTION

The disclosure describes examples of medical systems, devices, and techniques for creating a fluid pathway, or shunt, between a left atrium and a right atrium of a mammalian heart. The shunt can be maintained without the use of an implant, e.g., such as a stent. The shunt can be used, for example, to treat patients having heart failure and/or pulmonary edema.

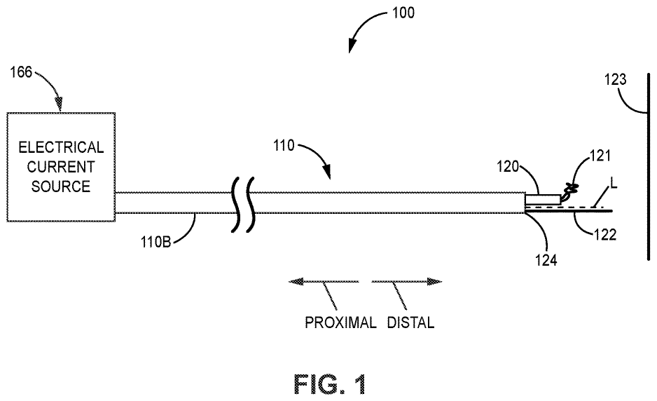

FIG. 1 is a perspective view depicting an example medical device 100 configured to form a shunt at a target treatment site of a heart. In some examples described herein, the medical device 100 includes a catheter 110 defining one or more lumen, and one or more inner members configured to be received in the lumen of catheter 110. The one or more inner members are configured to extend distally outward from a distal end 124 of the catheter 110.

The one or more inner members comprise an elongated support member 122, e.g., a guidewire, configured to move axially within the catheter 110 lumen, where the elongated support member 122 defines a longitudinal axis L. In some examples, the catheter 110 is made of latex, silicon, and/or Teflon. The catheter 110 can be a multi-lumen catheter (e.g., wire 121 and elongated support member 122 have separate lumens), or a single-lumen catheter may be used. In some examples, support member 122 may be advanced to a target location for formation of a shunt, e.g., the interatrial wall, prior to catheter 110. In such examples, catheter 110 may be advanced to the target location over support member 122.

The one or more inner members may also include an electrosurgical device 120 comprising at least one electrically conductive wire 121 having an active portion. The active portion may comprise an uninsulated portion of the wire 121 configured to ablate tissue of a septal wall 123 when an electrical current is applied to the wire 121, for example, by an electrical current source 166. The active portion is configured to provide a cut, so as to form a single flap comprising a biostable valvular shunt in the septal wall 123. In some examples, the active portion may form a biostable U-shaped, V-shaped, C-shaped, horseshoe-shaped, parabolic-shaped, square-shaped, or arc-shaped valvular shunt in the septal wall 123. For example, the wire 121 can be folded and/or bent to provide the U-shaped valvular shunt. In some examples, the U-shaped valvular shunt may form a flap. It is possible to separately advance and/or retract the wire 121 relative to the device 120, to change the size of the flap. Elongated support member 122 is configured to puncture the septal wall 123 to provide stability for placement of the active portion of the wire 121 against the septal wall 123. In some examples, a distal portion of support member 122 may be configured to assume a non-linear shape, e.g., curled shape, on an opposite side of septal wall 123 to, for example, prevent support member 122 from backing out of septal wall 123.

In some examples, the U-shaped, V-shaped, C-shaped, horseshoe-shaped, parabolically-shaped, square-shaped, or arc-shaped valvular shunt is rendered biostable by inhibiting overgrowth, scarring, and/or reattachment of a removed portion of the ablated tissue of the septal wall 123 to reduce and/or prevent the valvular shunt from closing. In the process of wound healing, tissue overgrowth of the septal wall 123 may occur that leads to a fusing together and closing of the valve. Accordingly, the ablation of the septal wall 123 by the wire 121 may be performed so as to cause scarring, lesions, or the like, that prevent the tissue from fusing together and closing the valvular shunt. In some examples, at least a portion of the valvular shunt can be biostabilized for a period of time, e.g., until one or more underlying causes of a pressure differential between the left and right atria are removed and/or eliminated, and the valvular shunt is no longer needed. For example, after a period of time, the pressure differential between the left and right atria may reduce, and one or more biostabilized leaflets, tissue flaps, and/or cuspids formed from the ablated septal wall 123 may be in contact for longer periods of time, which may promote fusing. In other examples, after the period of time and reduction of the pressure differential, surgical and/or pharmaceutical treatments may be used to further promote fusing of the valvular shunt.

In some examples, the electrical current source 166 is configured to deliver one or more electrical pulses using at least one of radiofrequency energy, microwave energy, or pulsed electric field energy to ablate the septal wall 123. In some examples, the electrical current source 166 may be a signal generator configured to generate radio-frequency energy at one or more frequencies in an approximate frequency range from 100 KHz to 1000 KHz, and at an RF output power level in an approximate range of 1 watt to 100 watts. In a further example, the electrical current source can be a Covidien ValleyLab Force 2 Electrosurgical Unit configured for a cut mode at 510 KHz, with an RF output power level of approximately 10 watts. In other examples, the RF output power may be adjusted based upon a gauge or diameter of the wire 121, a size and/or a dimension of the active portion of the wire 121, and/or a presence of a radiopaque element. In some examples, the active portion comprises an uninsulated portion of an insulated wire that transfers the electrical current to the septal wall 123. Wire 121 that directs the electrical pulse against the septal wall 123 may be provided in any of a variety of sizes (e.g., small, medium, and/or large-gauged wire). In one example, a diameter or gauge of the wire 121 may fall within a size range of guidewire diameters used for interventional procedures (for example, 0.035″=19 gauge, to 0.014″=27 gauge). In a further example, the electrical current comprises one or more electrical pulses, and a magnitude of the one or more pulses is based on a thickness of the septal wall 123. In some examples, in lieu of or in addition to using the insulated wire 121, the one or more inner members may function as an electrically insulating material. In some examples, the wire 121 can move axially in the one or more inner members.

In some examples, a size of the active portion of the wire 121 can be adjusted through the catheter 110 proximally, such that at least one dimension of the U-shaped, V-shaped, C-shaped, horseshoe-shaped, parabolically-shaped, square-shaped, arc-shaped, or other similarly-shaped, valvular shunt is adjusted. For instance, the active portion of the wire 121 may be varied in size by pushing/pulling on the wire from a proximal end 110B of the catheter 110. In one example, a handle with a lever may be provided at the proximal end 110B of the catheter 110. When pushed forward, the lever may extend a loop formed by the active portion of the wire 121. In a further example, the lever is a simple 1:1 push lever which, when pushed fully forward, completely extends the loop. Thus, the size of the active portion of the wire 121 can be adjusted by adjusting a length of a loop forming the active portion of the wire 121 from the proximal end 110B of the catheter 110. In a further example, the catheter 110 may have an outer diameter in a range of 7 French to 12 French, and the inner member may comprise an inner catheter having an outer diameter range of 4 French to 8 French.

In some examples, the septal wall 123 is an atrial septal wall between a left atrium and a right atrium of a mammalian heart. A size of the active portion of the wire 121 is determined based on a blood-pressure gradient between the left atrium and the right atrium. In some examples, a larger active portion of the wire 121 may be used when the blood pressure gradient is larger.

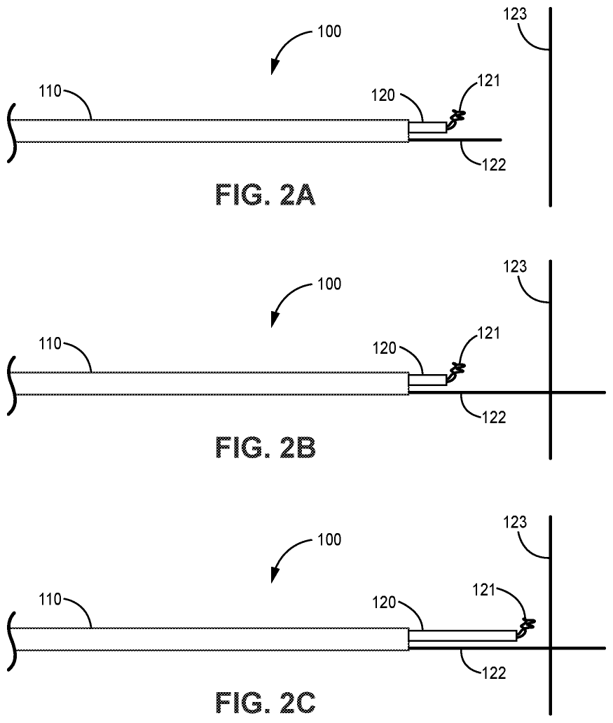

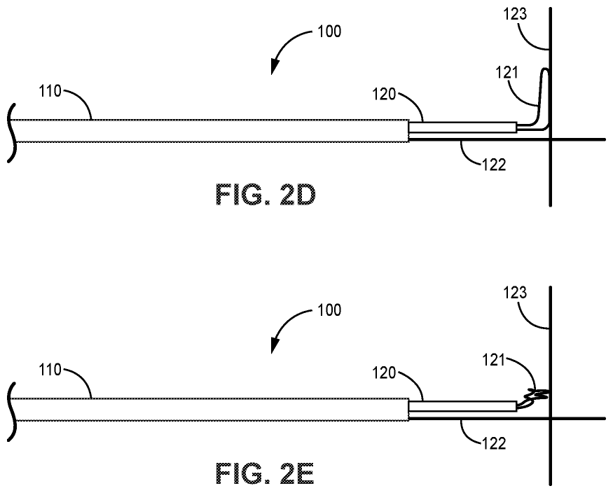

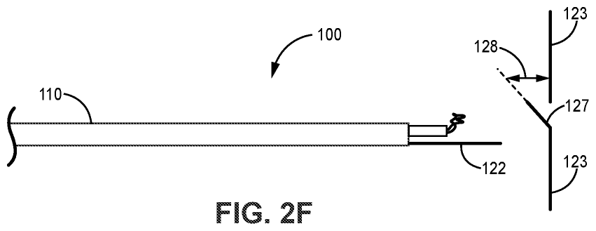

In some examples, at least one of the elongated support member 122 or the active portion of the wire 121 comprises a nitinol material. In some examples, an electrically insulating material is used to insulate at least a portion of the wire 121. In a further example, the electrically insulating material may comprise any of polyethylene, polyurethane, another plastic, nylon, PTFE, PEP, and/or another fluoropolymer. In some examples, at least one of the elongated support member 122 or the active portion of the wire 121 comprises a radiopaque material for providing a surface that is viewable on a fluoroscopy image, so as to facilitate a placement of the elongated support member and/or the active portion of the wire 121 against the septal wall 123. In some examples, a radiopaque marker portion of the wire 121 may be provide near a top, apex, or arch of the active portion of the wire 121 to ensure that a loop formed by the active portion is extended and positioned against a septal wall 123 (FIG. 2D). Likewise, the radiopaque marker portion may enable a physician to visualize that the loop burned through the septal wall 120 and was in a horizontal orientation (see FIG. 2E). In some examples, the radiopaque marker portion of the wire 121 may be formed using any of Gold, Platinum, Platinum-Iridium, or any of various combinations thereof. Gold, Platinum, and Platinum-Iridium are very radiopaque materials, and may have sufficient melting points to maintain integrity during ablation.

FIGS. 2A-2F illustrate an example technique for forming a biostable valvular shunt between a right atrium and a left atrium of a mammalian heart using the medical device of FIG. 1. FIG. 2A depicts a scenario where the medical device 100 is being advanced towards the septal wall 123, but is not yet in contact with the septal wall 123. Next, in FIG. 2B, the elongated support member 122 punctures the septal wall 123, but the active portion 121 has not yet come into contact with the septal wall 123. In FIG. 2C, the active portion 121 is brought into contact with the septal wall 123. Then, in FIG. 2D, the medical device is further advanced towards the septal wall 123, causing the active portion 121 to deflect against the septal wall. At FIG. 2E, electrical energy is now delivered to the active portion 121, ablating a portion of the septal wall 123 to provide a U-shaped valvular shunt. At FIG. 2F. the U-shaped valvular shunt forms a flap 127 leaning to the left of the septal wall 123. An angle 128 between the septal wall 123 and the flap 127 may be determined based on a pressure gradient, and/or based on an amount of flow. As the angle 128 increases, the flow also increases. In some examples, the wire can be sent through the septal wall and ablate the U-shape while pulling the wire proximally.

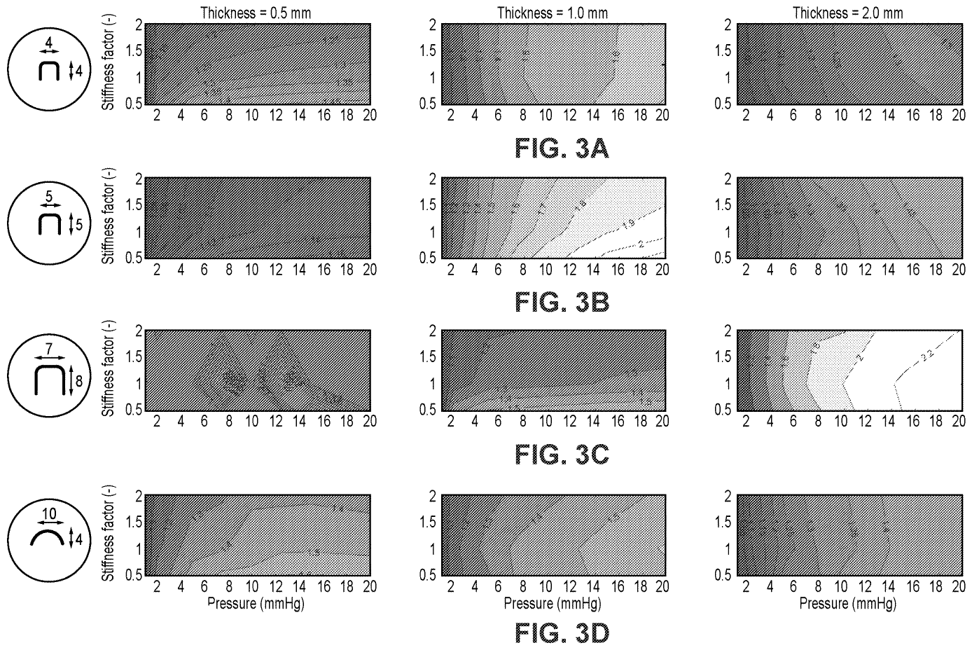

FIGS. 3A-3D comprise a set of graphs showing illustrative relationships between open cross-sectional area versus pressure gradient and material thickness for each of a plurality of biostable valvular shunts according to example embodiments. The set of graphs correspond to four different shapes for the biostable valvular shunt, while considering different septal wall thicknesses. An output variable of the graphs is a normalized cross-sectional area for each of the biostable valvular shunts, wherein the normalized cross-sectional area is indicative of how much the available shunting area changes with pressure.

Baseline cross-sectional area is assumed at 1 mmHg, and is used to normalize the cross-sectional area over a full range of assessment (1-20 mmHg). The resulting values for normalized cross-sectional area run from 1.0 (dark gray) to values as high as 2.2 (white) in some cases. In the examples of FIGS. 3A-3D, the shunting area available may increase by as much as a factor of 2.2, with such an increase directly impacting flow through the valvular shunt.

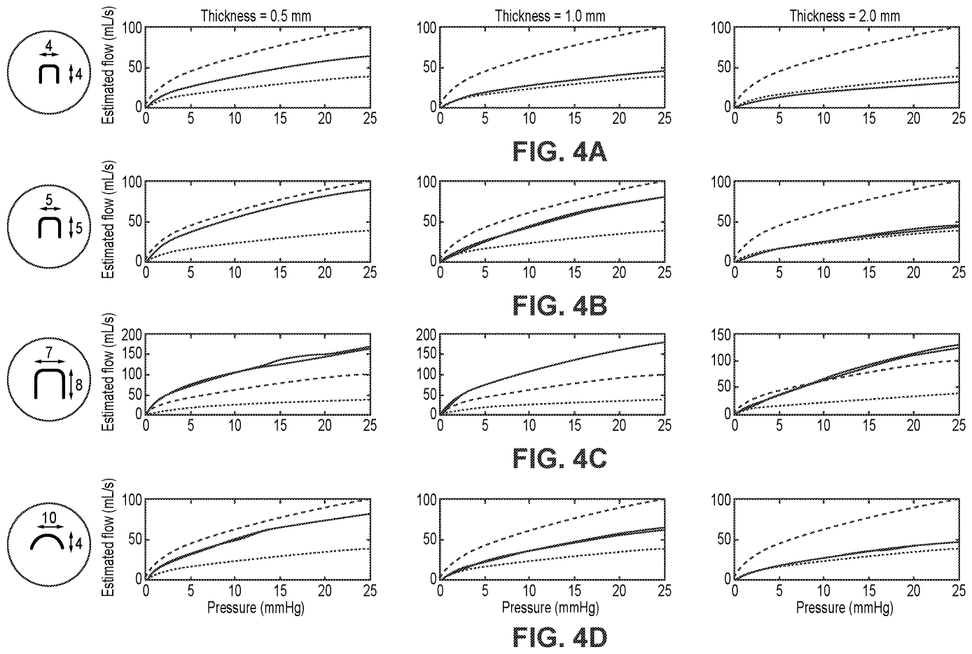

The graphs at the far left of FIGS. 3A-3D and 4A-4D show an illustrative pressure gradient for a septal wall 123 (FIG. 1) thickness of 0.5 mm, the middle graphs show an illustrative pressure gradient for a septal wall 123 thickness of 1.0 mm, and the graphs at the far right show an illustrative pressure gradient for a septal wall thickness 123 of 2.0 mm. The flow profile provided by the valvular shunts shown in FIGS. 3A-3D can be compared against a hypothetical flow profile provided by circular holes with fixed diameters, as shown in FIGS. 4A-4D s. These diameters can be selected based upon approximate bounding values for the size of the valvular shunt to be provided. In some examples, fixed-diameter circular holes of 5 mm and 8 mm can be used to provide a useful benchmark for comparing each of the plots of FIGS. 4A-4D.

FIG. 3A shows an illustrative relationship between open cross-sectional area versus pressure gradient and material thickness for a biostable valvular shunt with a U-shaped shunt size of 4 mm by 4 mm. FIG. 4A is a set of flow curves showing a projected flow profile for the valvular shunt of FIG. 3A compared to fixed circular holes with diameters of 5 mm and 8 mm. Similarly, FIG. 3B shows an illustrative relationship between open cross-sectional area versus pressure gradient and material thickness for a biostable valvular shunt with a U-shaped shunt size of 5 mm by 5 mm, and FIG. 4B is a set of flow curves showing a projected flow profile for the valvular shunt of FIG. 3B compared to fixed circular holes with diameters of 5 mm and 8 mm. Likewise, FIG. 3C shows an illustrative relationship between open cross-sectional area versus pressure gradient and material thickness for a biostable valvular shunt with a U-shaped shunt size of 7 mm by 8 mm, and FIG. 4C is a set of flow curves showing a projected flow profile for the valvular shunt of FIG. 3C compared to fixed circular holes with diameters of 5 mm and 8 mm. Similarly, FIG. 3D shows an illustrative relationship between open cross-sectional area versus pressure gradient and material thickness for a biostable valvular shunt with a parabolic/arc-shaped shunt size of 10 mm by 4 mm, and FIG. 4D is a set of flow curves showing a projected flow profile for the valvular shunt of FIG. 3D compared to fixed circular holes with diameters of 5 mm and 8 mm.

In some embodiments, for a given patient with a given septal wall 123 (FIG. 1) thickness, one may adjust the size and/or the shape of the valvular shunt to produce a desired flow profile. The desired flow profile may be based on a clinical recommendation, and/or the desired flow profile may be a one-size-fits-all. In one example, an amount of flow of the valvular shunt is designed to be between a first amount of flow provided by the fixed 5 mm diameter hole, and a second amount of flow provided by the fixed 8 mm diameter hole. In some embodiments, the shunt shapes shown in FIGS. 3A-3D and 4A-4D are self-regulating and adaptable. For example, when pressure differences increase, a flap formed by the U-shaped and/or parabolic/arc-shaped valvular shunt opens, and the shunting area increases.

When pressure normalizes, the valvular shunt closes. FIGS. 3A-3D and 4A-4D illustrate that, over a wide range of physiological septal wall thicknesses, 0.5 mm to 2.0 mm, and material properties, any of the different shunt shapes may open and close and regulate their area with respect to pressure. In some examples, atrial wall thickness may be used as a determinant to choose shunt size.

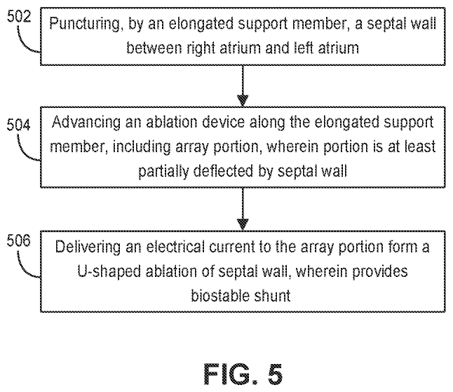

FIG. 5 is a flowchart illustrating an example technique for forming a biostable valvular shunt between a right atrium and a left atrium of a mammalian heart using the medical device of FIG. 1. At block 502, the elongated support member 122 (FIG. 1) punctures the septal wall 123. At block 504 (FIG. 5), a catheter carrying electrosurgical device 120 (FIG. 1) is advanced along the elongated support member 122, and the electrosurgical device may be advanced from the catheter toward the septal wall. The electrosurgical device 120 includes the active portion 121. The active portion 121 is at least partially deflected by the septal wall 123. At block 506 (FIG. 5), an electrical current is delivered to the active portion 121 (FIG. 1) to form a U-shaped ablation of the septal wall 123, wherein the U-shaped ablation provides a biostable shunt.

In accordance with example medical systems, devices, and techniques described herein, septal wall 123 tissue may be ablated by delivering electrical energy (e.g., radiofrequency (RF) energy, an electrical current, an electrical voltage, or the like), to the septal wall 123. Due to the nature of the ablation, the tissue adjacent to the ablation may fibrose/endothelialize and define an opening (e.g., a shunt) which may be formed as a multi-cuspid valve between the left atrium and the right atrium, enabling pressure from the left atrium to decompress into the right atrium. This may help treat heart failure and/or pulmonary edema, such as by mitigating a mechanism of heart failure and/or pulmonary edema. In other examples, the systems, devices, and techniques described herein can be used to create a shunt between two other hollow anatomical structures of a patient and to treat other patient conditions. Thus, while a shunt between a left atrium of a heart of a patient and a right atrium of the patient is primarily referred to herein, the systems, devices, and techniques can be used to form shunts in other locations of the heart, other locations of the body of patients, or for other medical procedures in other examples.

Interatrial shunting is currently being studied to treat heart failure and left atrial hypertension. All of the implant and non-implant based devices create a shunt with a single lumen diameter. Patients experience different left atrial (LA)-right atrial (RA) pressure differentials depending on their activity (sleep, rest, walking, exercising). Preliminary modeling illustrated how shunts of varying shapes could provide different flow rates between the LA and RA depending on LA-RA pressure gradients. Informed by this modeling, the techniques of this disclosure include creating a single-flap shunt in the atrial septal wall using an electrical current. In some embodiments, the single-flap shunt includes a U shape, a V shape, a C shape, a horseshoe shape, a parabolic shape, and/or an arc shape.

One feature of this disclosure is a device which electrosurgically creates the single-flap shunt in the atrial septal wall. The single-flap shunt has the advantage of being “self-regulating” with different left atrium-right atrium pressure gradients, depending on the dimensions of the shunt, as described in FIGS. 3A-3D and 4A-4D. The single-flap shunt can be used to provide an access point on the septal wall 123 for a large diameter therapy like a mitral replacement catheter.

The techniques of this disclosure can be used to treat pulmonary edema. For instance, forming a shunt between the left atrium and the right atrium with the systems and devices described herein enable the relief of fluid build-up in the lungs of a patient without requiring the permanent implantation of a foreign object (e.g., a stent or the like), leading to better patient outcomes. In addition, the systems and devices described herein are highly user-friendly, e.g., do not require extensive training for the clinician.

In some examples, this disclosure describes a method of creating a biostable shunt between a left atrium and a right atrium of a mammalian heart. The method includes puncturing, by an elongated support member, an atrial septum between the right atrium and the left atrium; advancing an ablation device along the elongated support member, the ablation device including an active portion, wherein the active portion is at least partially deflected by the atrial septum; and delivering an electrical current to the active portion to form a single-flap ablation of the atrial septum, wherein the single-flap ablation provides the biostable shunt. In some examples, the single-flap ablation is rendered biostable by inhibiting overgrowth, scarring, and/or reattachment of a removed portion of the ablated atrial septum to reduce and/or prevent the biostable shunt from closing.

The following examples are illustrative of the techniques described herein.

Example 1: A medical device including: a catheter defining a lumen; and an electrosurgical device movable within the lumen and configured to extend distally outward from a distal end of the catheter, the electrosurgical device including at least one electrically conductive wire having an active portion configured to cut septal wall tissue when an electrical current is applied to the wire; wherein the active portion is configured to provide a cut so as to form a single flap including a biostable valvular shunt in the septal wall tissue.

Example 2: The medical device of example 1, further including an elongated support member configured to move axially within the catheter lumen, the elongated support member defining a longitudinal axis, wherein the elongated support member is configured to puncture the septal wall tissue to provide stability for placement of the active portion against the septal wall tissue.

Example 3: The medical device according to any of the preceding examples, wherein the electrical current is configured to deliver one or more pulses by at least one of radiofrequency energy, microwave energy, or pulsed electric field energy to ablate the septal wall tissue.

Example 4: The medical device according to any of the preceding examples, wherein a size of the active portion can be adjusted through the catheter proximally, such that at least one dimension of the valvular shunt is adjusted.

Example 5: The medical device according to any of the preceding examples, wherein the electrical current includes one or more pulses, and a magnitude of the one or more pulses is based on a thickness of the septal wall tissue.

Example 6: The medical device according to any of examples 2-5, wherein at least one of the elongated support member or the active portion includes a nitinol material.

Example 7: The medical device according to any of examples 2-6, wherein at least one of the elongated support member or the active portion includes a radiopaque material for providing a surface that is viewable on a fluoroscopy image.

Example 8: The medical device according to any of the preceding examples, wherein the catheter is less than or equal to a 7 French sized catheter and the electrosurgical device is less than or equal to a 5 French sized inner catheter.

Example 9: The medical device according to any of the preceding examples, wherein the electrosurgical device includes an inner catheter configured to insulate at least a portion of the wire.

Example 10: The medical device according to any of the preceding examples, wherein the active portion includes an uninsulated portion of an insulated wire that transfers the electrical current to the septal wall tissue.

Example 11: The medical device according to any of the preceding examples, further including an insulated structure configured for enclosing at least a portion of the wire, and configured for allowing an adjustment of a size of the active portion by moving the wire relative to the insulated structure.

Example 12: A method of creating a biostable shunt in a septal wall between a right atrium and a left atrium of a heart, the method including: advancing an electrosurgical device towards the septal wall, the electrosurgical device including an active portion, wherein the active portion is at least partially deflected by the septal wall; and delivering an electrical current to the active portion to provide an ablation of the septal wall from a cut, so as to form a single flap including the biostable shunt.

Example 13: The method of example 12, further including: puncturing, by an elongated support member, the septal wall between the right atrium and the left atrium; and advancing the electrosurgical device along the elongated support member towards the septal wall.

Example 14: The method according to examples 12 or 13, wherein the delivering of the electrical current further includes delivering one or more pulses by at least one of radiofrequency energy, microwave energy, or pulsed electric field energy to ablate the septal wall.

Example 15: The method according to any of examples 12-14, further including determining a size of the active portion based on one or more of: a blood-pressure gradient between the left atrium and the right atrium; or a thickness of the septal wall.

Example 16: The method according to any of examples 12-15, wherein the delivering of the electrical current further includes: determining a thickness of the septal wall, and delivering one or more pulses; and a magnitude of the one or more pulses is based on the thickness of the septal wall.

Example 17: The method according to any of examples 13-16, wherein at least one of the elongated support member or the active portion is formed using a nitinol material.

Example 18: The method according to any of examples 13-17, wherein at least one of the elongated support member or the active portion is formed using a radiopaque material for providing a surface that is viewable on a fluoroscopy image, so as to facilitate a placement of the elongated support member and/or the active portion against the atrial septum.

Example 19: The method according to any of examples 12-18, wherein the active portion is formed with an uninsulated portion of an insulated wire that transfers the electrical current to the septal wall.

1. A medical device comprising:
a catheter defining a lumen; and
an electrosurgical device movable within the lumen and configured to extend distally outward from a distal end of the catheter, the electrosurgical device comprising at least one electrically conductive wire having an active portion configured to cut septal wall tissue when an electrical current is applied to the wire;
wherein the active portion is configured to provide a cut so as to form a single flap comprising a biostable valvular shunt in the septal wall tissue.

2. The medical device of claim 1, further comprising an elongated support member configured to move axially within the catheter lumen, the elongated support member defining a longitudinal axis, wherein the elongated support member is configured to puncture the septal wall tissue to provide stability for placement of the active portion against the septal wall tissue.

3. The medical device according to claim 1, wherein the electrical current is configured to deliver one or more pulses by at least one of radiofrequency energy, microwave energy, or pulsed electric field energy to ablate the septal wall tissue.

4. The medical device according to claim 1, wherein a size of the active portion can be adjusted through the catheter proximally, such that at least one dimension of the valvular shunt is adjusted.

5. The medical device according to claim 1, wherein the electrical current comprises one or more pulses, and a magnitude of the one or more pulses is based on a thickness of the septal wall tissue.

6. The medical device according to claim 2, wherein at least one of the elongated support member or the active portion comprises a nitinol material.

7. The medical device according to claim 2, wherein at least one of the elongated support member or the active portion comprises a radiopaque material for providing a surface that is viewable on a fluoroscopy image.

8. The medical device according to claim 1, wherein the catheter is less than or equal to a 7 French sized catheter and the electrosurgical device is less than or equal to a 5 French sized inner catheter.

9. The medical device according to claim 1, wherein the electrosurgical device comprises an inner catheter configured to insulate at least a portion of the wire.

10. The medical device according to claim 1, wherein the active portion comprises an uninsulated portion of an insulated wire that transfers the electrical current to the septal wall tissue.

11. The medical device according to claim 1, further comprising an insulated structure configured for enclosing at least a portion of the wire, and configured for allowing an adjustment of a size of the active portion by moving the wire relative to the insulated structure.

12. A method of creating a biostable shunt in a septal wall between a right atrium and a left atrium of a heart, the method comprising:
advancing an electrosurgical device towards the septal wall, the electrosurgical device including an active portion, wherein the active portion is at least partially deflected by the septal wall; and
delivering an electrical current to the active portion to provide an ablation of the septal wall from a cut, so as to form a single flap comprising the biostable shunt.

13. The method of claim 12, further comprising:
puncturing, by an elongated support member, the septal wall between the right atrium and the left atrium; and
advancing the electrosurgical device along the elongated support member towards the septal wall.

14. The method of claim 12, wherein the delivering of the electrical current further comprises delivering one or more pulses by at least one of radiofrequency energy, microwave energy, or pulsed electric field energy to ablate the septal wall.

15. The method of claim 12, further comprising determining a size of the active portion based on one or more of: a blood-pressure gradient between the left atrium and the right atrium; or a thickness of the septal wall.

16. The method of claim 12, wherein the delivering of the electrical current further comprises:
determining a thickness of the septal wall, and
delivering one or more pulses; and
a magnitude of the one or more pulses is based on the thickness of the septal wall.

17. The method of claim 13, wherein at least one of the elongated support member or the active portion is formed using a nitinol material.

18. The method of claim 13, wherein at least one of the elongated support member or the active portion is formed using a radiopaque material for providing a surface that is viewable on a fluoroscopy image, so as to facilitate a placement of the elongated support member and/or the active portion against the atrial septum.

19. The method of claim 12, wherein the active portion is formed with an uninsulated portion of an insulated wire that transfers the electrical current to the septal wall.
